(19) United States
(12) Patent Application Publication
(10) Pub. No.: US 2018/0333076 A1
(43) Pub. Date: Nov. 22, 2018

(54) BREATHING TUBE FOR USE IN LUNG FUNCTION DIAGNOSTICS

(71) Applicant: ndd Medizintechnik AG, Zurich (CH)

(72) Inventor: Erich KLEINHAPPL, Waedenswil (CH)

(21) Appl. No.: 15/598,175

(22) Filed: May 17, 2017

(51) Int. Cl. A61B 5/097; A61B 5/091; A61B 5/087

(52) U.S. Cl. CPC A61B 5/097; A61B 5/087; A61B 5/091

(57) ABSTRACT

A breathing tube for use in lung function diagnostics comprises a breathing tube body having first and second windows. The first window allows ultrasonic waves to pass from an exterior to an interior of the breathing tube body and vice versa, and the second window allows ultrasonic waves to pass from an interior to an exterior of the breathing tube body and vice versa. First and second sealing elements circumferentially extend around the breathing tube body on its outside and define a first breathing tube body section located between the first and second sealing elements, in which the first and second windows are located. At least one of the first and second sealing elements includes a first groove, a second groove, and a sealing lip located between the first groove and the second groove, which protrudes over a surface of the breathing tube body.

### BACKGROUND

The instant disclosure relates in an aspect to a breathing tube for use in lung function diagnostics, the breathing tube having improved sealing capabilities.

US 2016/0128608 A1 describes a breathing tube for use in lung function diagnostics having a sealing lip. This sealing lip directly projects from the surface of the breathing tube. The sealing lip needs to have a sufficiently big height in order to achieve a good sealing of a section of the breathing tube against its surrounding. However, it turned out that big forces need to be applied in order to insert the breathing tube into a corresponding lung function diagnostics device and to remove it again from that device after use. In order to allow an easy operation of the breathing tube, it would be desirable if the sealing lip would be smaller. Then, however, worse sealing properties would result.

### SUMMARY

It is an object of the instant disclosure to provide a breathing tube that has at least the same sealing properties for a section of the breathing tube, but can be more easily inserted and removed from a lung function diagnostics device.

This object is achieved by a breathing tube having the features explained in the following.

Such a breathing tube is intended to be used in lung function diagnostics in connection with lung function diagnostics devices.

The term “lung function diagnostics” refers to any kind of the analysis of breath gas (i.e., the analysis of gas inhaled or exhaled by a person) to determine the lung function of a patient, in particular all applications of spirometry, gas washout measurements, gas dilution measurements, or gas diffusion measurements. Typical parameters determined by lung function diagnostics are forced vital capacity (FVC), forced expiratory volume in 1 second (FEV1), FEV1/FVC ratio (FEV1%), forced expiratory flow (FEF), forced inspiratory flow 25-75% or 25-50%, peak expiratory flow (PEF), tidal volume (TV), total lung capacity (TLC), diffusing capacity (DLCO), maximum voluntary ventilation (MVV), functional residual capacity (FRC), and/or lung clearance index (LCI). The instantly described and/or claimed breathing tube is intended to be used for determining any of these parameters in spirometry or to be used for any other kinds of lung function diagnostics without specific limitation.

The breathing tube comprises a breathing tube body having an outside and an inside. The outside of the breathing tube body faces the inside of a breathing tube receptacle of a lung function diagnostics device when the breathing tube is inserted into a corresponding lung function diagnostics device. In operation, gas is exhaled or inhaled through a gas flow space that is surrounded by the inside of the breathing tube body.

The breathing tube comprises a first window being arranged on a first side of the breathing tube body. The first window is designed and arranged to allow ultrasonic waves to pass from an exterior of the breathing tube to an interior of the breathing tube (i.e., to the gas flow space). Likewise, ultrasonic waves can pass from an interior of the breathing tube to an exterior of the breathing tube through the first window.

The breathing tube further comprises a second window being located on a second side of the breathing tube body. Thereby, the second side is opposite the first side of the breathing tube. The second window is designed and arranged to allow ultrasonic waves to pass from an interior of the breathing tube body to an exterior of the breathing tube body and vice versa.

If ultrasonic waves are directed onto the breathing tube, they can enter the interior of the breathing tube through the first window, pass through the interior of the breathing tube and can then exit the breathing tube through the second window. Likewise, ultrasonic waves can enter the breathing tube through the second window, pass through its interior and then exit the breathing tube through the first window.

In order to reduce any gas flow through the windows from the interior of the breathing tube to an exterior thereof, the first window and/or the second window are typically closed by a gauze-like mesh. But still then, portions of gas flowing through the interior of the breathing tube can pass through the first window or the second window from the interior of the breathing tube to an exterior thereof. As already explained above, the exterior of the breathing tube is, in operation, surrounded by a breathing tube housing of a lung function diagnostics device.

In the area in which the windows of the breathing tube are arranged, ultrasonic transceiver housings are located in the corresponding lung function diagnostics device. If gas could flow out of the interior of the breathing tube through the first window and/or the second window it could also flow into the ultrasonic transceiver housings and into other parts of the lung function diagnostics device into which the breathing tube is inserted in operation. In order to restrict a corresponding flow, the first window and the second window are located in a first section of the breathing tube body that is encompassed by the first sealing element and the second sealing element. The first sealing element and the second sealing element circumferentially extend around the outside of the breathing tube body transverse a flow direction at a distance between the first sealing element and the second sealing element. This distance defines the size of the first breathing tube body section that is located between the first sealing element and the second sealing element. This section needs to be dimensioned such that the first window and the second window can be located within the first breathing tube body section.

By designing the first sealing element and/or the second sealing element such that it comprises a first groove, a second groove and a sealing lip located between the first groove and the second groove, good sealing properties with low mechanical resistance for introducing the breathing tube into a lung function diagnostics device or removing it from a corresponding device can be achieved. For this purpose, the sealing lip projects from the wall of the breathing tube body and protrudes over the surface of the breathing tube body.

In contrast to prior art solutions according to which a simple sealing lip has been placed onto the surface of the breathing tube, the sealing element of the instantly claimed breathing tube with the sealing lip arranged in between two grooves provides for very good sealing properties combined with a significantly lower mechanical resistance than “classic” sealing elements known from prior art. Specifically, in an embodiment, it was possible to reduce the force being necessary to insert the breathing tube into a holding device of a lung function diagnostics device and to remove it therefrom by 50% compared to a breathing tube with a usual sealing lip. Therewith, a corresponding sealing element is particularly appropriate to be used in connection with a breathing tube having a non-circular cross-section, such as a quadrangular cross-section.

To allow for a particular easy introducing of the breathing tube into a lung function diagnostics device and removal of the breathing tube from a corresponding lung function diagnostics device, both the first sealing element and the second sealing element comprise, in an embodiment, in each case a first groove, a second groove, and a sealing lip located between the first groove and the second groove. Thereby, the sealing lip protrudes over a surface of the breathing tube body, as explained above.

In an embodiment, the first groove is directly adjacent to the sealing lip on a first side of the sealing lip. Likewise, the second groove is in this embodiment directly adjacent to the sealing lip on a second side of the sealing lip being opposite to the first side. Expressed in other words, the first groove passes into the sealing lip, wherein the sealing lip itself passes into the second groove. Such an arrangement allows for a high flexibility of the sealing lip, thereby making it particularly easy to insert the breathing tube into a lung function diagnostics device or to remove it from such a device.

In an embodiment, the first groove and the second groove have identical dimensions, i.e., they are constructed in the same way. This allows for identical bending properties of the sealing lip upon inserting the breathing tube into a lung function diagnostics device and removing it from such a device.

In an embodiment, the height of the grooves is small in comparison to the height of the sealing lip. I.e., a first distance between the surface of the breathing tube body and the lowest point of the first groove or the lowest point of the second groove is smaller than a second distance between the surface of the breathing tube body and the highest point of the sealing lip. It turned out that comparatively small grooves are sufficient to allow for high flexibility of the sealing lip. To achieve good sealing properties, the sealing lip should nonetheless have a height sufficiently big to compensate for any constructional size variations of the breathing tube and to allow in any case for good sealing properties.

In an embodiment, the sealing lip has a base portion which passes into the breathing tube body. Thus, the sealing lip is integrally formed with the breathing tube body, e.g., by injection molding. Furthermore, the sealing lip has a top portion defining a free end of the sealing lip. Thereby, the base portion has a bigger width than the top portion. Thus, in cross-section, the sealing lip has a cone-like appearance. The top portion of the sealing lip is intended to abut the inner surface of a breathing tube receiving space of a lung function diagnostics device. If the sealing lip has, in cross-section, a cone-like appearance, such abutment can be achieved in a particularly appropriate manner.

In an embodiment, the width dimensions of the grooves and of the sealing lip are adjusted to each other. Thereby, the sealing lip has at its half height (being the half between the end of the base portion and the end of the top portion of the sealing lip) a width that is smaller than a width of the first groove and/or the second groove.

In an embodiment, the breathing tube or the breathing tube body comprises at least one plastic chosen from the group consisting of polyethylene (PE), polypropylene (PP), acrylonitrile butadiene styrene (ABS), polyether ether ketone (PEEK), polycarbonates (PC), polystyrene (PS), polyethylene terephthalate (PET), polyethylene terephthalate glycol (PETG), polyamide (PA), polyacetal (polyoxymethylene, POM), as well as blends and copolymers thereof. In an embodiment, the breathing tube or the breathing tube body essentially consists of only a single material (which can be a copolymer of different plastic materials or a composite material comprising reinforcing elements). A copolymer of PE and PP is particularly appropriate.

In an embodiment, a mesh covering a window of the breathing is provided. Such a mesh can comprise or can be made entirely of a material that is identical or different from the material used for the flow tube body. Appropriate materials for the mesh are polyesters, PA, PET, PP, chlorotrifluoroethylene (CTFE), ethylene tetrafluoroethylene (ETFE), as well as blends and copolymers thereof. In an embodiment, the mesh essentially consists of only a single material (which can be a copolymer of different plastic materials or a composite material comprising reinforcing elements).

Since it was able to construct the sealing lip in a more flexible manner by the provision of two grooves positioned next to the sealing lip, the whole breathing tube can be made from a material that has higher stability or higher rigidity than materials used in prior art for producing breathing tubes. To give an example, high-density polyethylene (HDPE) can well be used for producing a breathing tube according to the instant disclosure.

If an overall harder material is used for producing the breathing tube than the materials used in prior art for producing breathing tubes, a breathing tube results that is more resistant to bites. This, in turn, means that the cross-section of a corresponding breathing tube is not significantly altered even if a patient bites on it during breath analysis. This results in a high accuracy of a measurement which is performed by using a corresponding breathing tube.

Thus, the breathing tube described herein allows for higher accuracy in lung function diagnostics and higher ease of use due to several reasons. First, harder materials than used in prior art can be used. Therewith, the occurrence of deformations of the breathing tube due to bites of a patient are reduced. Second, a good sealing of a section of the breathing tube to be sealed against the environment is achieved due to providing a specially designed sealing element. Third, an easy inserting of the breathing tube into a lung function diagnostics device and removing the breathing tube from a lung function diagnostics device is made possible due to low mechanic resistance of the sealing element.

All embodiments explained in the preceding sections can be combined in any desired way and any desired combination and sub-combination.

### BRIEF DESCRIPTION OF THE DRAWINGS

Further details of aspects of the instant disclosure will be explained with respect to an exemplary embodiment and accompanying Figures. In the Figures:

FIG. 1 is a perspective view of an embodiment of a breathing tube;

FIG. 2 is a cross-section through the wall of the breathing tube of FIG. 1 in the area of a sealing element; and

FIG. 3 is a perspective detail view of the sealing element of the breathing tube of FIG. 1.

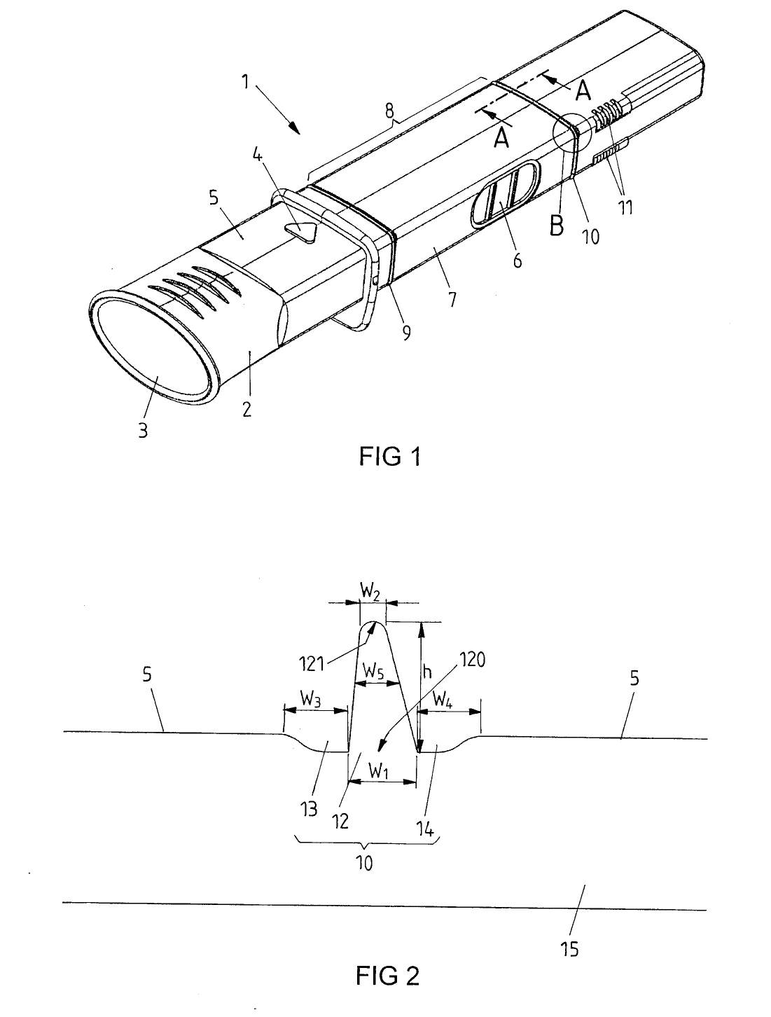

FIGS. 1-3 are shown drawn approximately to scale. However, other relative dimensions may be used if desired.

### DETAILED DESCRIPTION

FIG. 1 shows a breathing tube 1 with an integrated mouthpiece 2 through which gas can be exhaled into an interior 3 of the breathing tube 1 or can be inhaled from this interior 3. Thus, the interior 3 serves as gas flow space.

The breathing tube 1 is intended to be inserted into a lung function diagnostics device. To allow for a correct orientation of the breathing tube 1 within such a lung function diagnostics device, an indicator 4 is provided on a surface 5 of the breathing tube 1.

The breathing tube 1 comprises a first window 6 through which ultrasonic waves can be transmitted into the interior 3 of the breathing tube 1. The first window 6 is located on a first side 7 of the breathing tube 1 Likewise, a second window is located on a second side lying opposite to the first side 7. In the depiction of FIG. 1, neither the second side nor the second window can be seen.

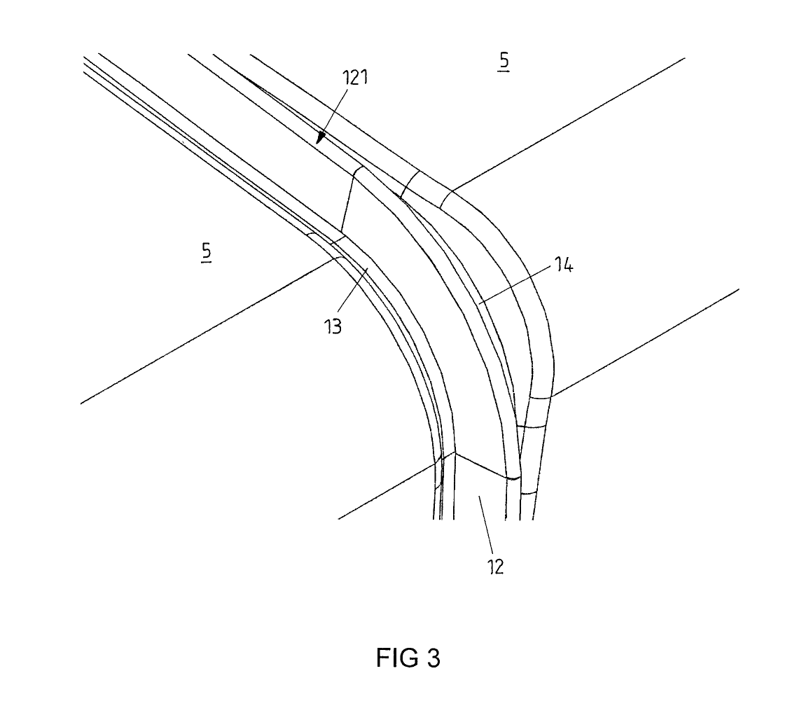

The first window 6 as well as the second window are located in a sealed section 8 of the breathing tube 1 that is limited on its first side by a first sealing arrangement 9 and on its second side by a second sealing arrangement 10. The sealing arrangements 9, 10 serve as sealing elements. The first sealing arrangement 9 and the second sealing arrangement 10 comprise in each case a sealing lip extending circumferentially around an outside of the breathing tube 1 transverse a gas flow direction. Thereby, the second sealing arrangement 10 is constructed in the same manner like the first sealing arrangement 9. More details with respect to the sealing arrangements 9, 10 will be explained with respect to FIG. 2 and FIG. 3.

In a section of the breathing tube 1 located distally from the mouthpiece 2, a coding structure 11 in form of a comb-like structure is provided on a first and on a second of the edges of the breathing tube 1. This coding structure 11 can be read out by a light source in order to identify the type of the breathing tube 1 and its correct positioning within a lung function diagnostics device.

FIG. 2 shows a schematic cross-section through the sealing arrangement 10 along the line A-A in FIG. 1. In this and the following Figure, the same elements will be referred to with the same numeral references as in the preceding Figure(s).

It can be seen that a sealing lip 12 is located between a first groove 13 and a second groove 14. Thereby, the sealing lip 12, the first groove 13 and the second groove 14 make up the sealing arrangement 10. To be more specifically, a first section of the surface 5 of the breathing tube passes into the first groove 13 which, in turn, passes into the sealing lip 12 which, in turn, passes into the second groove 14 which, in turn, passes into another section of the surface 5 of the breathing tube.

Thereby, the sealing lip 12 projects from the wall 15 of the breathing tube and protrudes over the surface 5 of the breathing tube. A base portion 120 of the sealing lip 12 is located at a transition between the wall 15 and the sealing lip 12. A top portion 121 of the sealing lip 12 makes up a free end of the sealing lip 12. The base portion has a width w₁ that is bigger than a width w₂ of the top portion 121. Since the first groove 13 is constructed identically to the second groove 14, its width w₃ is identical to the width w₄ of the second groove 14. The distance from the base portion 120 to the top portion 121 of the sealing lip 12 defines its height h. At half of this height h, the sealing lip 12 has a width ws which is smaller than the width w₃ of the first groove 13 or the width w₄ of the second groove 14.

FIG. 3 shows a detail view of an area of the breathing tube 1 that is encircled and marked with “B” in FIG. 1. It shows the transition of a first section of the surface 5 of the breathing tube to the first groove 13, the transition of the first groove 13 to the sealing lip 12, as well as the second groove 14 and its transition to another section of the surface 5 of the breathing tube 1.

The sealing capability of the breathing tube illustrated in FIGS. 1 to 3 has been examined. This breathing tube was found to have a leakage rate of 3100 hPa*s/1 when inserted into a lung function diagnostics device. I.e., when a pressure of 3100 hPa was applied to the interior of the breathing tube, a flow of 1 liter gas per second from one side of the sealing lip to the other side of the sealing lip was observed.

In contrast, a prior art breathing tube having identical dimensions but a sealing lip without adjacent grooves (Comparative Sealing Tube) showed a leakage rate of 2400 hPa*s/1 under identical measuring conditions. Thus, a flow of 1 liter gas per second was already observed at an applied pressure of 2400 hPa.

Consequently, the sealing arrangement of the breathing tube illustrated in FIGS. 1 to 3 showed a sealing capability that was approximately 30% higher than the sealing capability of the sealing lip of the prior art breathing tube. At the same time, the force being necessary to insert the breathing tube of FIGS. 1 to 3 into a holding device of a lung function diagnostics device and to remove it therefrom was reduced by 50% compared to the Comparative Sealing Tube.

FIGS. 1-3 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What claimed is:

1. A breathing tube for use in lung function diagnostics, comprising:
a breathing tube body;
a first window located on a first side of the breathing tube body, the first window serving for allowing ultrasonic waves to pass from an exterior of the breathing tube body to an interior of the breathing tube body and vice versa;
a second window located on a second side of the breathing tube body, wherein the second side is opposite the first side, the second window serving for allowing ultrasonic waves to pass from an interior of the breathing tube body to an exterior of the breathing tube body and vice versa;
a first sealing element and a second a sealing element, wherein the first sealing element and the second a sealing element circumferentially extend around the breathing tube body on its outside and thereby define a first breathing tube body section located between the first sealing element and the second sealing element;
wherein the first window and the second window are located within the first breathing tube body section;
wherein at least one of the first sealing element and the second sealing element comprises a first groove, a second groove, and a sealing lip located between the first grove and the second groove, the sealing lip protruding over a surface of the breathing tube body.

2. The breathing tube according to claim 1, wherein the first sealing element and the second sealing element in each case comprise a first groove, a second groove, and a sealing lip located between the first grove and the second groove, the sealing lip protruding over a surface of the breathing tube body.

3. The breathing tube according to claim 1, wherein the first groove and the second groove are directly adjacent to the sealing lip.

4. The breathing tube according to claim 1, wherein the first groove and the second groove have identical dimensions.

5. The breathing tube according to claim 1, wherein a first distance between the surface of the breathing tube body and a lowest point of the first groove or the second groove is smaller than a second distance between the surface of the breathing tube body and a highest point of the sealing lip.

6. The breathing tube according to claim 1, wherein the sealing lip has a base portion at which it passes into the breathing tube body and a top portion defining a free end of the sealing lip, wherein the base portion has a bigger width than the top portion.

7. The breathing tube according to claim 6, wherein a width of the sealing lip at its half height is smaller than a width of the first groove or the second groove.

8. The breathing tube according to claim 1, wherein it comprises at least one plastic chosen from the group consisting of polyethylene and polypropylene.

9. The breathing tube according to claim 8, wherein it comprises a high-density polyethylene.
